(19) United States
(12) Patent Application Publication
(10) Pub. No.: US 2023/0404689 A1
(43) Pub. Date: Dec. 21, 2023

(54) MEDICAL ROBOTIC SYSTEM

(71) Applicant: Meditrina, Inc., San Jose, CA (US)

(72) Inventors: Csaba TRUCKAI, Saratoga, CA (US); John H. SHADDUCK, Menlo Park, CA (US); Akos TOTH, Cupertino, CA (US)

(73) Assignee: Meditrina, Inc., San Jose, CA (US)

(21) Appl. No.: 18/332,161

(22) Filed: Jun. 9, 2023

Related U.S. Application Data
(60) Provisional application No. 63/366,524, filed on Jun. 16, 2022.

(51) Int. Cl. A61B 34/30; A61B 1/018; A61B 17/32; A61B 1/303; A61B 8/12

(52) U.S. Cl. CPC A61B 34/30; A61B 1/018; A61B 17/32002; A61B 1/303; A61B 8/12; A61B 2034/301

(57) ABSTRACT

Robotic surgical systems configured to control the movement and actuation of a single robotic arm, and the movement and actuation of multiple tools carried at a distal end of the robotic arm.

### CROSS-REFERENCE TO RELATED APPLICATIONS

This application claims benefit of priority to U.S. Provisional Patent Application No. 63/366,524 filed Jun. 16, 2022, the content of which is incorporated herein by reference in its entirety.

### BACKGROUND

The present invention is related to a robotic surgical system that is configured to control the movement and actuation of a single robotic arm and the movement and actuation of multiple tools in a gynecology procedure.

### SUMMARY

The principles of the present disclosure relate to medical robotic systems. For example, some aspects of the techniques described herein relate to medical robot systems, including a robotic arm having a plurality of moveable arm segments; an endoscopic viewing assembly detachably coupled to a distal segment of the robotic arm, the endoscopic viewing assembly having an elongate endoscope shaft extending about a longitudinal axis to a distal end carrying an image sensor; and a stabilizing device detachably coupled to a distal segment of the robotic arm, the stabilizing device having an elongate tool shaft extending about a longitudinal axis adapted for engaging tissue to stabilize a cervix of a patient.

Variations of a medical robot system can further include a motor drive configured to manipulate various implements on the medical robotic system. For example, the motor drive can rotate the elongate endoscope shaft relative to its longitudinal axis. The motor drive can also be configured to move the elongate endoscope shaft axially relative to its longitudinal axis.

Additionally, the motor drive is configured to rotate the elongate tool shaft relative to its longitudinal axis. The motor drive can also be configured to move the elongate tool shaft axially relative to its longitudinal axis.

In some aspects, the techniques described herein relate to a medical robot system further including a treatment tool detachably coupled to a distal segment of the robotic arm, the treatment tool having a treatment tool shaft extending about a longitudinal axis to a working end, wherein the treatment tool shaft is configured for introduction through the working channel of the endoscopic viewing assembly.

Motor drives of the present robotic system can be configured to rotate the treatment tool shaft relative to its longitudinal axis and/or move the treatment tool shaft axially relative to its longitudinal axis. Examples of such treatment tools include but are not limited to, a resection device, ablation device, coagulation device, biopsy device, and dissection device.

In additional variations, the treatment tool includes a resection device with a moving cutting member, and wherein the system includes a resecting motor drive for moving, the moving cutting member moves at least rotationally or axially.

Variations of the medical robot system include a cervical canal sealing assembly coupled to at least one of the robotic arms and the endoscopic viewing assembly. In additional variations, a distal end of the cervical canal sealing assembly includes a cervical seal configured for movement co-axially with a medial portion of the elongate endoscope shaft.

Additionally, the present robotic system can include motor drives that are adapted for moving the cervical seal. Variations of the system include a contact sensor carried by the cervical seal adapted to sense contact with a cervix of the patient and send signals of the contact or lack thereof to a controller. In additional variations, the medical robot system includes a controller that is configured to actuate the motor drive to move the cervical seal responsive to the signals of the contact from the contact sensor. Variations of the system include a contact sensor that is either a pressure sensor, capacitance sensor, impedance sensor, and optical sensor. Alternatively, the contact sensor can include multiple contact sensors, including combinations of those listed above.

In some aspects, the techniques described herein relate to a method of treating tissue in a patient's uterine cavity, including: providing a medical robot system with robotic arm having a plurality of moveable arm segments with a distal arm segment carrying a plurality of motor drives for moving at least one device coupled to the distal arm segment; a resecting device detachably coupled to the distal arm segment, the resecting device having an elongate shaft extending about longitudinal axis to a working end carrying a cutter configured to rotate and/or axially reciprocate; introducing the working end of the resecting device transcervically into the patient's uterine cavity; actuating the resecting device to rotate and/or reciprocate the cutter; and utilizing a controller operates at least one of the plurality of motor drives to move the working end of the resecting device in a predetermined pattern to resect tissue.

The methods disclosed herein can further include a controller that operates a motor drive to move the working end in an axial pattern while actuating the cutter to resect tissue. In addition, the controller can operate a motor drive to move the working end in a rotational pattern while actuating the cutter to resect tissue.

The present disclosure is related to commonly assigned U.S. patent application Ser. No. 17/662,182, the entirety of which is incorporated by reference.

### BRIEF DESCRIPTION OF DRAWINGS

Additional aspects of the invention will become clear from the following description of illustrative embodiments and from the attached drawings, in which:

FIG. 1 is an illustration of a surgical robotic system, including a roll stand with a tower portion, a surgical console, and user input interface, and a surgical robotic arm adapted to carry and robotically manipulate multiple tools in procedures to treat intrauterine tissues.

FIG. 2 is an enlarged view of the robot arm of FIG. 1 with a single-use endoscopic viewing system and a single-use tissue resecting device detached from the robotic arm.

FIG. 3 is a view similar to FIG. 2, with the endoscopic viewing system and tissue resecting device locked into place in the robotic arm.

FIG. 4 is a perspective view of the proximal housing and the working end of the single-use endoscope of the endoscopic viewing system of FIGS. 1-3, wherein the endoscope is of the type that has an expandable and collapsible working channel in its working end.

FIG. 5 is a perspective view of the endoscopic viewing system of FIGS. 1-3 from a different angle of the proximal housing and the working end of the single-use endoscope, which has an expandable and collapsible working channel.

FIG. 6 is a perspective view of the working end of the endoscope of FIG. 4 with the shaft of a tubular cutter device inserted through the expandable-collapsible working channel of the endoscope and further showing a pre-selected or programmed robotic cutting pattern.

FIG. 7 is a perspective view of the proximal portion of a resection device that comprises a drive component and tubular cutter, where only the proximal housing of the tubular cutter is shown.

FIG. 8 is a perspective view of the tubular cutter of FIG. 7 with an enlarged view of the working end showing the rotating inner sleeve and cutting windows.

FIG. 9 is a perspective view of the proximal portions of the endoscopic viewing system and the resection device assembled as when detachably coupled to the robotic arm as in FIGS. 1 and 3.

FIG. 10 is a perspective view of the proximal portion of a robotic tissue-stabilizing assembly with its drive component adjacent to the endoscopic viewing system and resection device of FIG. 9.

FIG. 11 is a perspective view of a tenaculum component of the tissue-stabilizing assembly of FIG. 10.

FIG. 12 is a perspective view of a cervical seal component that is slidably mounted over the shaft of the endoscope.

FIG. 13 is a perspective view of a robotically operated cervical seal assembly that is slidably mounted over the shaft of the endoscope.

FIG. 14 is a perspective view of the proximal portion of the robotic cervical seal assembly of FIG. 13, shown assembled with the tissue-stabilizing assembly, endoscopic viewing system, and resection device of FIG. 10.

FIG. 15 is a perspective view of the distal working ends of the endoscope, the cervical seal, the tubular cutter, and the tenaculum, indicating the dimension of robotic movements of some of the components.

FIG. 16A is a schematic sectional view of a patient's uterus showing a step in a method of using the robotic arm to move a tenaculum-type into position to grasp the patient's cervix and move an endoscope toward the patient's cervix.

FIG. 16B is a schematic view of a subsequent step of the method illustrating the actuation of the robotic arm to advance the endoscope working end through the endocervical canal into the uterine cavity and also the movement of a cervical seal toward the patient's cervix.

FIG. 16C illustrates another subsequent step of the method where the robotic arm is actuated to advance the resection device through the working channel of the endoscope into the uterine cavity and further actuating the robotic arm to angle the working end of the tubular cutter towards a targeted fibroid, and also showing a cervical seal advanced to contact the cervix.

FIG. 16D illustrates another subsequent step of the method with the robotic arm moving the working end of the tubular cutter laterally further to resect the fibroid.

FIG. 17 is a perspective view of the working ends of the endoscope and the tubular cutter showing a pre-selected or programmed robotic cutting pattern.

FIG. 18 is a perspective view of the working end of an endoscope similar to that of FIGS. 1-14 with another variation of a treatment tool comprising a retractor and a resection device where both the retractor and resection device are dimensioned for introduction through the working channel of the endoscope.

FIG. 19 is an enlarged perspective view of the working end of the retractor of FIG. 18.

FIG. 20 is a sectional view of the endoscope shaft, the retractor shaft, and the tubular cutter shaft taken along line 20-20 of FIG. 18.

FIG. 21A is a side view of the endoscope shaft with the retractor and tubular cutter of FIG. 18 extended outwardly from the working channel.

FIG. 21B is a side view of a variation of a retractor and the tubular cutter of FIG. 18 extended outwardly from the working channel.

FIG. 22 is a schematic view of a method of using the retractor of FIG. 8 to engage and stabilize a polyp in a uterine cavity for resection.

FIG. 23 is a perspective view of an endoscope together with another variation of a treatment tool comprising a grasper device that is used to grasp and stabilize a polyp.

FIG. 24A is a view of the working end of the grasper device of FIG. 23 with jaws in an open position.

FIG. 24B is a view of the working end of the grasper of FIG. 24A with the jaws in a closed position.

FIG. 25 is a sectional view of the shaft or the grasper device of FIG. 23 in the working channel of the endoscope alongside the shaft of the tubular cutter.

FIG. 26 is an enlarged perspective view of the working end, another aspiration-based retractor similar to the variation of FIG. 18 with an internal actuator for adjusting the shape of the working end.

FIG. 27 is a schematic view of the working end of another grasper with an internal actuator for closing the jaw structure.

FIG. 28 is a schematic view of the working end of a grasper with an external sleeve for closing the jaw structure.

FIG. 29 is a perspective view of the working end of an endoscope with another variation of a treatment tool comprising an ultrasound device that can be introduced through the working channel together with a tubular cutter.

FIG. 30 is a schematic view of another variation of an ultrasound device that is positioned on the exterior of the patient's body.

### DETAILED DESCRIPTION

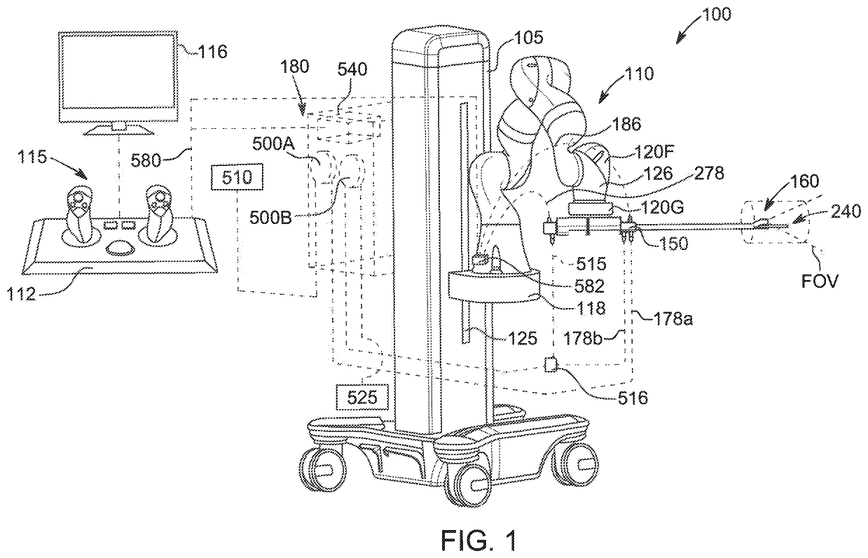

FIG. 1 illustrates a surgical robotic system 100 adapted for use in gynecology procedures or other procedures with similar access requirements that includes a roll stand 104 with a vertical column or tower 105 that carries a surgical robot assembly comprising a robotic arm 110 that is operated from a console 112 with user input interface 115 and image display 116. The user input interface 115 can comprise one or more joysticks, rollerballs, and other input mechanisms known in the art. The image display 116 may be a touch screen that can further be used to direct movement of the arm 110 and/or control other operating parameters of the system 100 as described below. The robotic arm 100 is capable of movement relative to multiple axes as provided by multiple drives and actuators. In one variation, a robotic arm 110 of the system 100 can comprise as a core unit a commercially available, multiple-segment robotic arm manufactured by KUKA Robotics Corporation, having an office at 51870 Shelby Parkway, Shelby Township, Michigan, 48315.

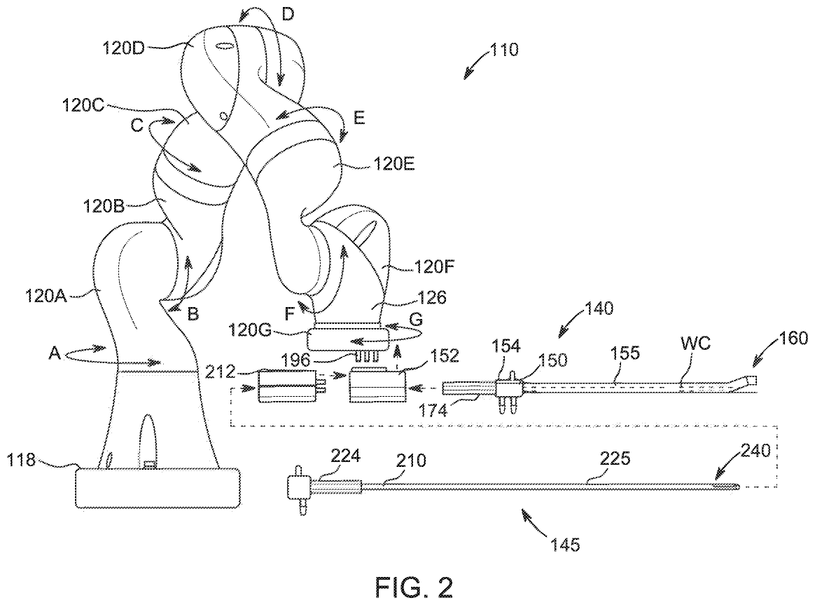

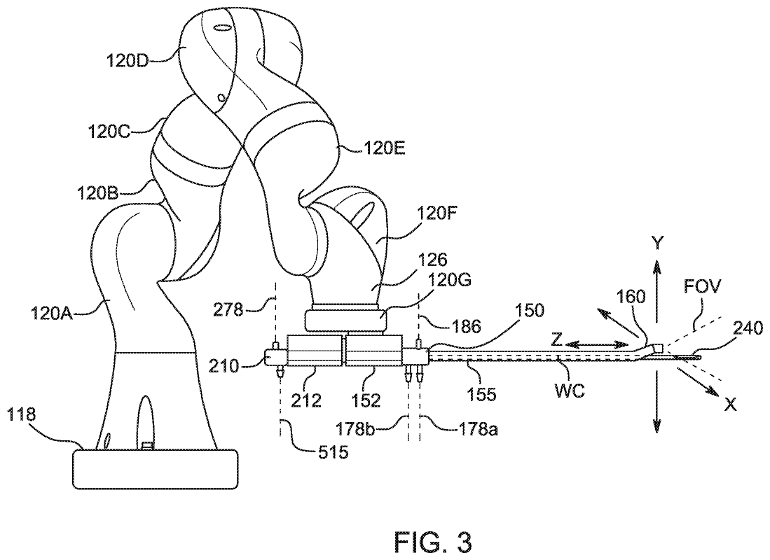

Referring to FIGS. 1 and 2, the lower base platform 118 of robotic arm 110 is motor driven to move vertically on a vertical rail 125 within the tower 105 of the roll stand 104. In a variation, the robotic arm 110 is configured with seven moveable arm segments 120A-120G, with the base rotational arm segment 120A rotating relative to base platform 118 (FIG. 2). The arm segments 120A-120G are adapted to rotate as indicated by arrows A through G in FIG. 2. The distal end 126 of the sixth arm segment 120F is coupled to the seventh rotating segment 120G that is motor driven to rotate. As will be described below, additional motor drives and surgical tools are adapted for detachable coupling to rotating arm segment 120G. Referring to FIG. 3, in a surgical procedure, it can be understood that the robotic arm 110 can move the distal end of a tool in all directions, angles, pitch, and yaw, for example, directions X, Y, and Z.

Referring to FIGS. 2 and 3, it can be seen that the first and second cooperating tools are adapted for attachment to the rotating segment 120G. The first tool is a single-use endoscopic viewing assembly 140, and the second tool is a single-use tissue resecting device 145 configured for insertion through a working channel WC in the endoscopic viewing assembly 140. In FIG. 2, the endoscopic assembly 140 and the resecting device 145 are shown detached from the rotating segment 120G of the robotic arm. In FIG. 3, the endoscopic assembly 140 and the resecting device 145 are fully assembled with the robotic arm 110.

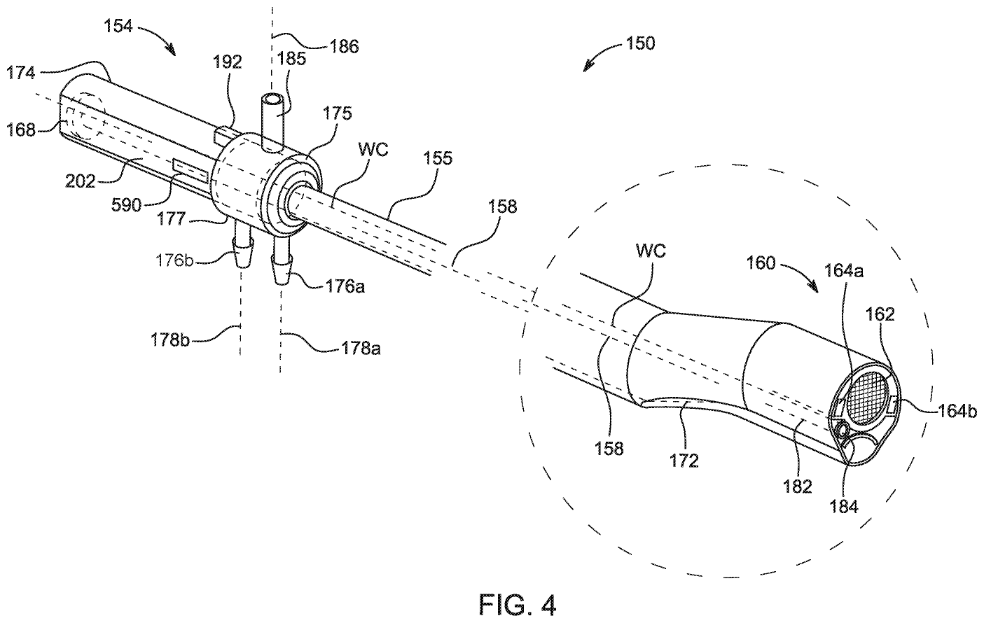

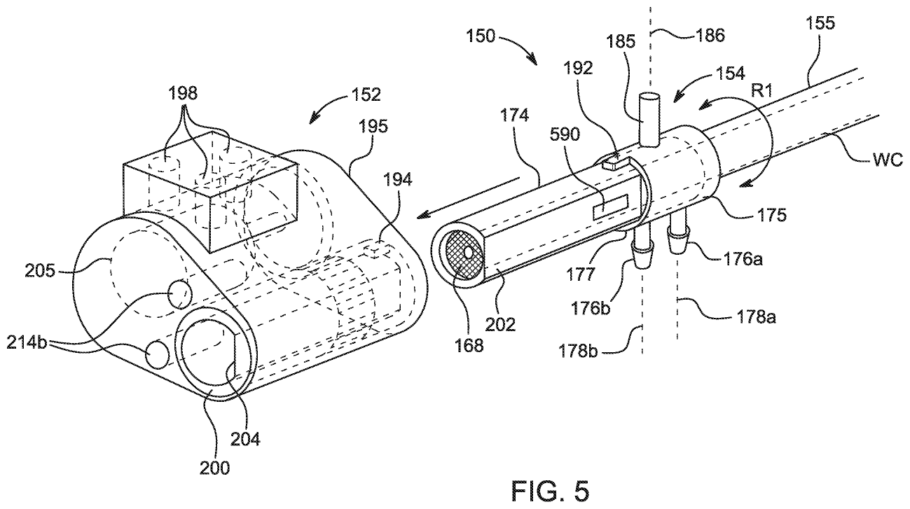

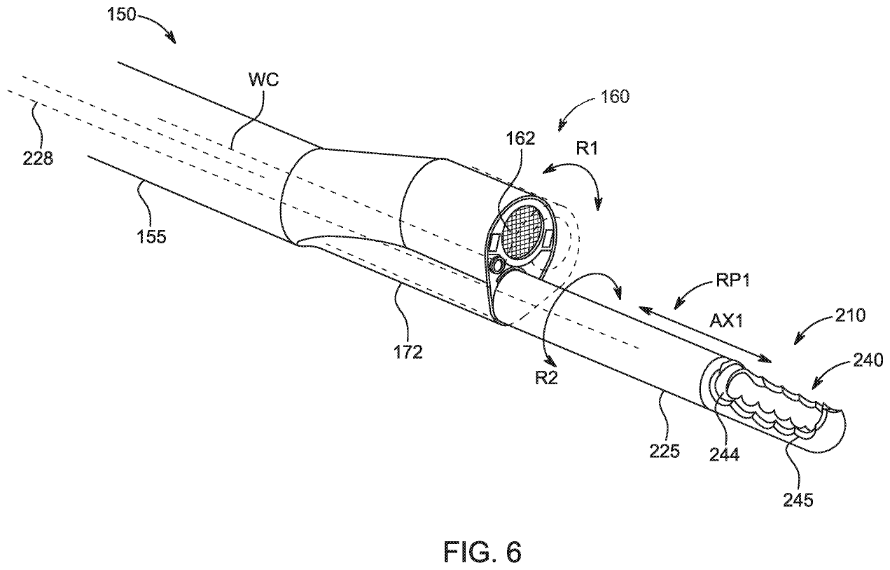

FIGS. 4 and 5 are enlarged illustrations of the endoscopic viewing assembly 140 that comprises an endoscope 150 (FIG. 4) and an endoscope drive component 152 (FIG. 5). The endoscope 150 has a proximal hub 154 coupled to an elongated shaft 155 extending about a longitudinal axis 158 to a working end 160 that carries an image sensor 162 and at least one LED with two LEDs 164a and 164b shown in this variation (FIG. 4). The endoscope 150 has a working channel WC with a proximal seal 168, wherein the working channel extends through the elongated shaft 155 to the working end 160 of the endoscope. In FIG. 4, it can be seen that the working end 160 of the endoscope has an S-shape or curve in a repose or insertion shape with a small cross-section that allows for atraumatic introduction through a patient's cervical canal. The working channel WC has a distal region 172 that is expandable in cross-section to receive the shaft of a component of the resecting device 145, shown in FIGS. 6, or to receive the shaft of any similar tool with a straight, rigid shaft. The working end 160 of the endoscope shaft 155 of FIG. 5 and other related endoscopes with expandable working channels and systems are described in more detail in commonly-owned U.S. Pat. Nos. 10,433,717 and 11,259,695 and commonly-owned U.S. patent application Ser. Nos. 15/975,626; 16/351,909; 16/562,069; 16/848,050; 17/447,380 and 17/490,643 which are incorporated herein by this reference.

Referring to FIGS. 4 and 5, it can be seen that the hub 154 of the endoscope 150 has a proximal extending portion 174 and a rotating collar 175 that carries two Luer fittings 176a and 176b in its inferior surface 177. Luer fitting 176a is adapted for coupling to the fluid inflow tubing 178a from a tubing set of a fluid management system 180, shown in FIG. 1 and described further below. The second Luer fitting 176b is adapted for coupling to outflow tubing 178b the fluid management system 180 (FIG. 1). The inflow tubing 178a communicates with an inflow channel 182 in the endoscope shaft 155 with an open termination 184 in the working end 170 (FIG. 4). The working channel WC in the endoscope shaft 155 functions as the fluid outflow channel and communicates with Luer fitting 176b and outflow tubing 178b. The arrangement of components in the interior of rotating collar 175 that allow for inflows and outflows from the rotatable endoscope shaft 155 to the rotating collar 175 is described in more detail in commonly-owned U.S. Pat. No. 10,433,717, which is incorporated herein by reference.

The superior surface of the rotating collar 175 includes a connector 185 for connecting an electrical cable 186 to the endoscope 150 to carry signals from the image sensor 162 to an image processor and to carry electrical current to LEDs 164a-164b. The rotating collar 175 includes a projecting key 192 that projects into a receiving notch 194 in the housing 195 of the drive component 152, which is adapted to maintain the rotating collar 175 in an upright position during use as the endoscope shaft 155 is rotated (see FIG. 5). The upright position of the collar 175 is useful for maintaining the inflow and outflow tubing 178a, 178b, and the electrical cable 186 (FIG. 1) in upright, non-rotating positions as the endoscope shaft 155 is rotated.

Now turning to FIG. 5, the endoscope drive component 152 may be adapted for single-use or multiple-use and typically would be adapted for multiple uses. In a variation, the drive component 152 detachably couples to arm segment 120G with pins 196 that are mated with bores 198 in the housing 195 of the drive component 152 (see FIG. 2).

Referring again to FIG. 5, the endoscope drive component 152 carries a rotating receiver 200 adapted to receive the proximal extending portion 174 of hub 154 of the endoscope 150. FIG. 5 shows that the proximal extending portion 174 has a keyed surface 202 that cooperates with keyed surface 204 of the rotating receiver 200, which provides for rotational locking of the endoscope 150 and drive component 152. The rotating receiver 200 is operatively coupled by a suitable gear mechanism to a motor drive 205 carried in the drive housing 195, and that is adapted to rotate the rotating receiver 200 and the endoscope 150 when locked in place in the direction of the arrow R1 in FIGS. 5 and 6. The motor drive 205 can be a DC stepper motor or other motor type and is adapted to rotate the rotating receiver 200 and locked-in place endoscope 150 from 180o to 360o. The gear mechanism can be any form of conventional straight gears that convert the motor shaft rotation to rotation of the receiver 200.

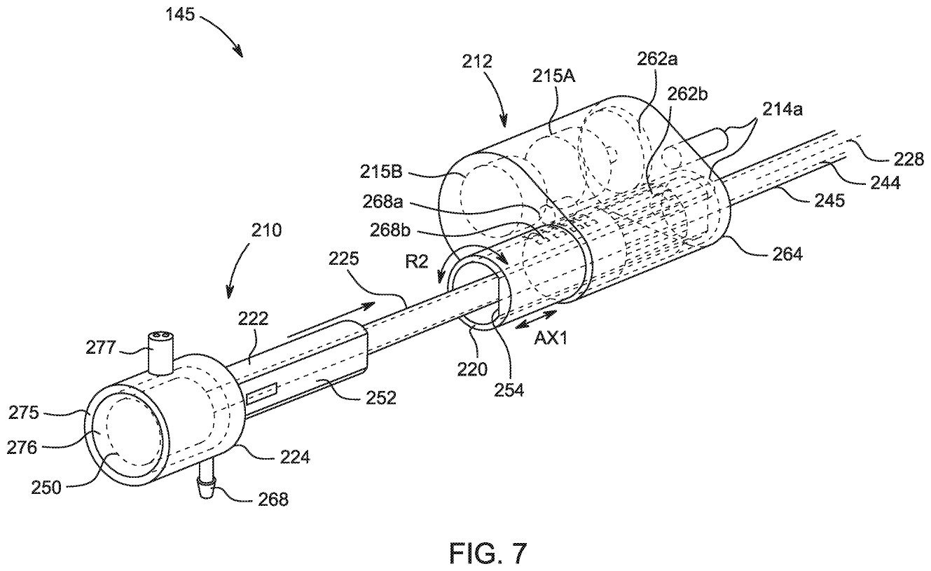

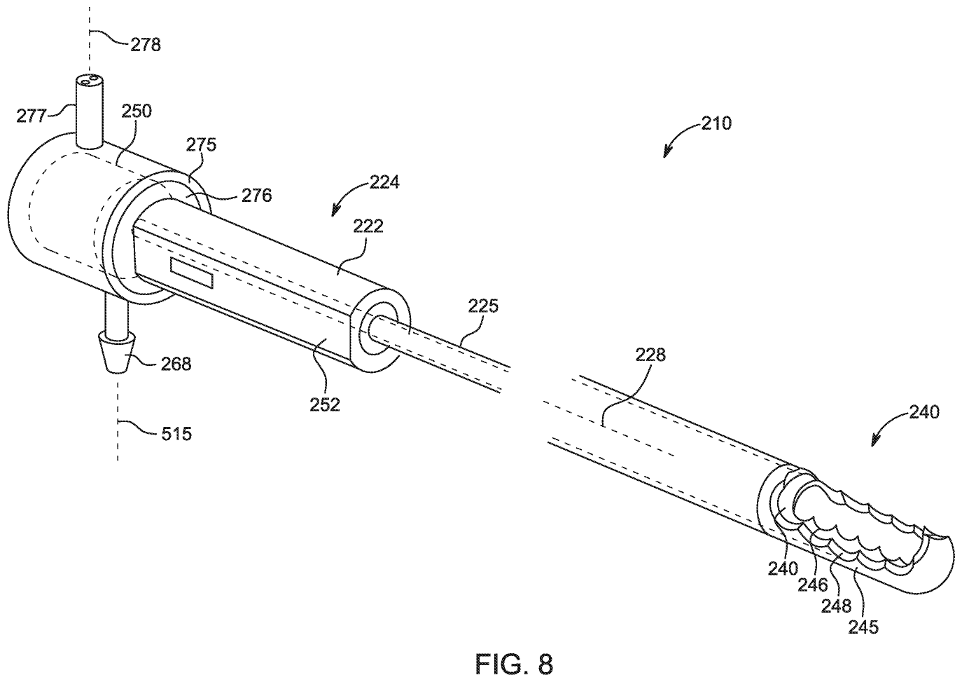

Now turning to FIGS. 7 and 8, the resection device 145 is illustrated and comprises a motor-driven tubular cutter 210 and a tool drive component 212. The drive component 212 may be adapted for single-use or multiple-use and typically would be adapted for multiple uses. In a variation, the tool drive component 212 detachably couples to housing 195 of the endoscope drive component with pins 214a that are mated with bores 214b in the endoscope drive housing 195 (FIG. 5). The tool drive component 212 carries first and second DC motors 215A and 215B for respectively (i) rotating and (ii) axially moving a moveable receiver 220 in the drive component 212 that receives an extending portion 222 of the proximal housing 224 of the tubular cutter 210.

As can be seen in FIG. 6, shaft 225 of the tubular cutter 210 is adapted for introduction through the working channel WC of the endoscope shaft 155 of FIGS. 4 and 5. It should be appreciated that other treatment tools can be used other than a tubular cutter, such as any type of ablation or coagulation device, RF device, biopsy device, ultrasound device, laser device, dissector, retractor, or manipulator, with the tubular cutter 210 shown for purposes of illustration.

Referring to FIGS. 7 and 8, the tubular cutter 210 has a proximal housing 224 coupled to the elongated shaft assembly 225 that extends about axis 228 to a working end 240 adapted for tissue resection (FIG. 8). In FIG. 8, it can be seen that shaft assembly 225 comprises concentric inner and outer sleeves 244 and 245, respectively, where the inner sleeve 244 has an inner cutting window 246 that rotates in an outer cutting window 248 of the outer sleeve 245 to resect tissue (see FIG. 8). The proximal housing 224 of the tubular cutter 210 carries a motor drive 250 for rotating the inner sleeve 244 and inner cutting window 246 at a suitable rotational speed ranging from 1,000 RPM to 10,000 RPM or more for cutting tissue.

Now referring to FIG. 7, the tubular cutter 210 is shown partially inserted into the tool drive component 212. The rotating receiver 220 of the drive component 212 is adapted to receive the extending portion 222 of the proximal housing 224 of the tubular cutter 210. FIG. 7 shows that the extending portion 222 of the housing 224 has a keyed surface 252 that cooperates with a keyed surface 254 of the rotating receiver 220 to provide for a locked-in rotational engagement of the proximal housing 224 and receiver 220. As can be understood, the shaft 225 of the tubular cutter 210, when assembled with other components as in FIG. 1-3 is further introduced through the seal 168 into the working channel WC of the endoscope 150.

In a variation, the rotating receiver 220 in FIG. 7 is operatively coupled to the first motor drive 215A by a gear mechanism that rotates the moveable receiver 220, for example, with geared surfaces 262a and 262b. The first motor drive, 215A, is adapted to rotate the receiver 220 and engaged cutter 210 from 180o to 360o. As indicated by arrows R2 in FIGS. 6 and 7. The tool drive component 212 further carries the second motor drive 215B that is geared to move the receiver 220 axially in the housing 264 of the drive component 212. A suitable gear mechanism can consist of a worm gear 268a that engages features 268b in the surface of the receiver 220. Thus, the second motor drive 215B is adapted to axially move the rotating receiver 220 (and engaged tubular cutter 210) back and forth in the drive housing 212 in the direction of arrow AX1 when the tubular cutter 210 is locked in place. The motor drives 215A and 215B again can be DC stepper motors or other suitable motor types. As can be understood, the second motor drive 215B is adapted to move the working end 240 of the tubular cutter 210 axially between a fully retracted position in the working channel WC of the endoscope 150 and a range of extended positions of the working end 240 as shown in FIG. 3 within the field of view FOV of the endoscope image sensor 162 during a procedure. The range of extension of the working end 240 beyond the endoscope 150 is up to 15 cm beyond the plane of the lens of the image sensor 162.

Referring to FIGS. 7 and 8, it also can be seen that the tubular cutter 210 is configured for use with the fluid management system 180. The fluid outflow tubing 178b of the fluid management system 180 is coupled to the Luer fitting 268 on the housing 224 of the tubular cutter 210. Fluid outflows and resected tissue is aspirated through the lumen of the inner sleeve 244 of the tubular cutter 210, as is known in the art. In FIGS. 7 and 8, it can be further seen that the proximal housing 224 of the tubular cutter 210 has a rotatable surface portion 275 that can freely rotate around a core portion 276. The arrangement of components in the interior of proximal housing 224 that allow for outflows from the tubular cutter 210 to communicate with the Luer fitting 268 in the rotatable surface portion 275 is described in more detail in commonly-owned U.S. Pat. No. 10,433,717, which is incorporated herein by reference. The rotatable surface portion 275 also carries a connector 277 for coupling with an electrical cable 278 to carry current to the motor 250 in the tubular cutter 210. The upright position of the rotatable surface portion 275 is useful for maintaining the outflow tubing 178b and the electrical cable 278 (FIG. 1) in stable, non-rotating positions.

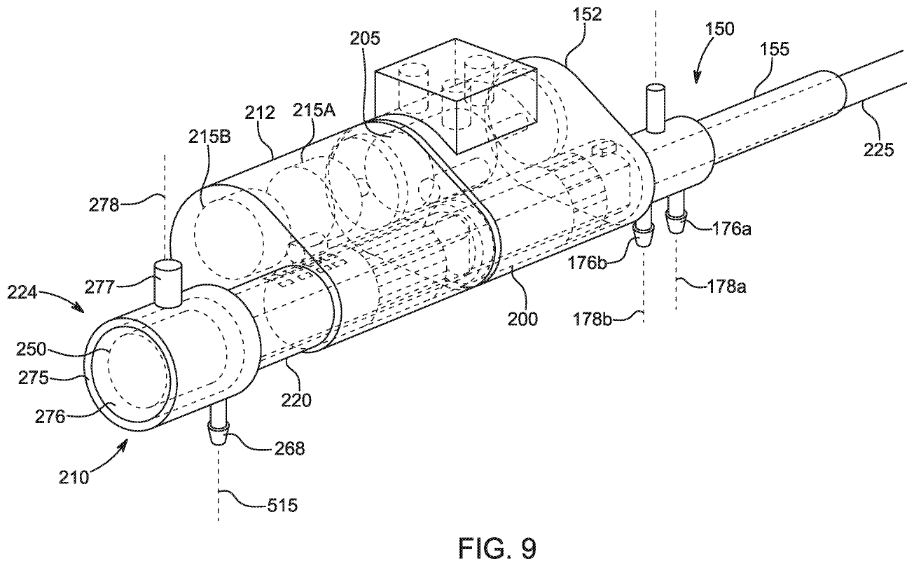

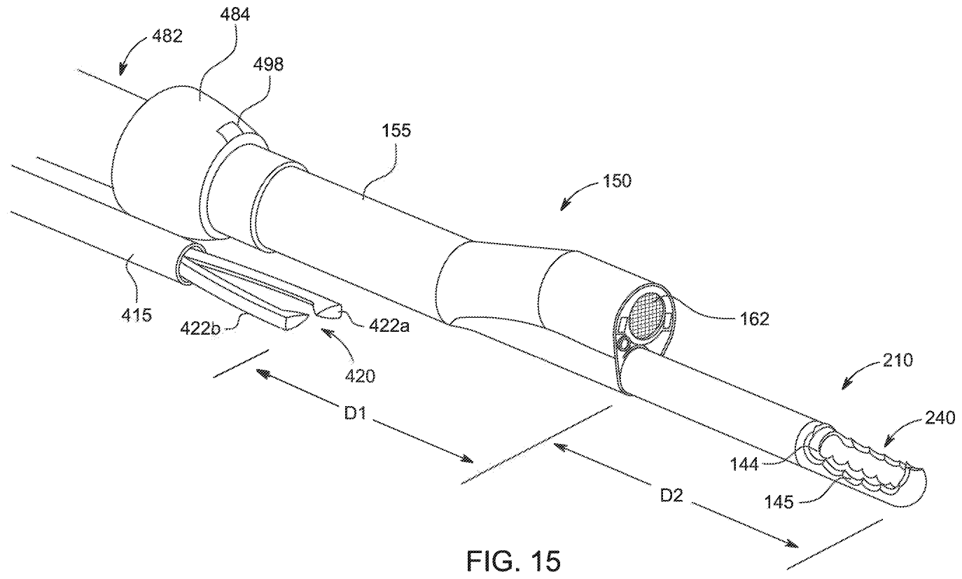

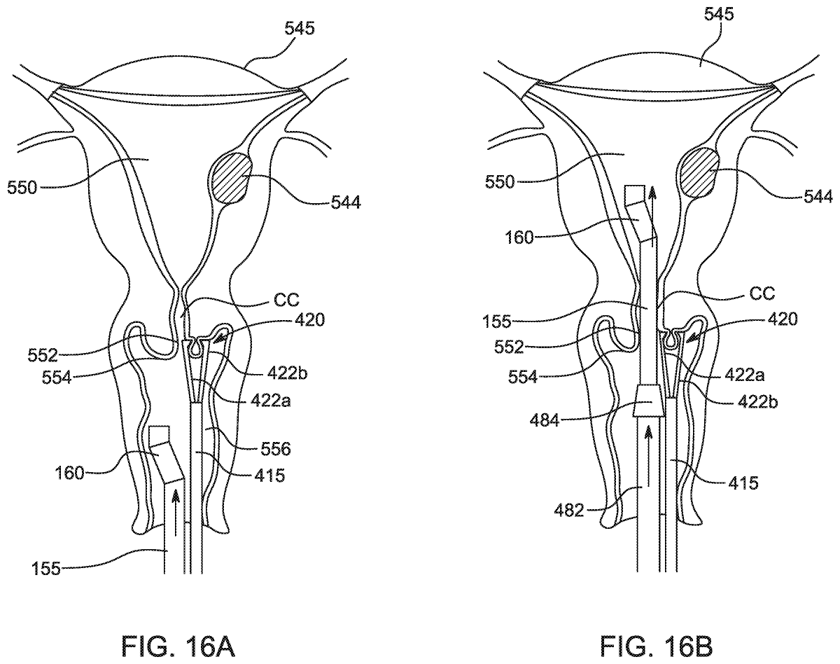

Now turning to FIG. 9, it can be seen how the endoscopic viewing assembly 140 and the resecting device 145 can be assembled and coupled to the rotating robotic arm segment 120G. The first drive component 152 is lifted vertically to couple to the pins 196 in the arm segment 120G. Thereafter, the extending portion 174 of the endoscope's proximal hub 154 is inserted into the receiver 200 of the endoscope drive component 152. Next, the tool drive component 212 and its pins 214a are mated with the receiving bores 214b in the endoscope drive housing 195. Finally, the elongated shaft 225 of the tubular cutter 210 is introduced through both drive components 152, 212 and the working channel WC of the endoscope 150. FIGS. 1 and 3 illustrate the shaft 225 and working end 240 of the tubular cutter 210, both fully extended beyond the working end 160 of the endoscope 150, as an example. However, it should be appreciated that when using the robotic arm 110, the endoscope shaft 155 is initially introduced through the patient's cervical canal CC (FIGS. 16A-16B) with the working end 240 of the tubular cutter 210 retracted within the working channel WC of the endoscope 150 as shown in FIGS. 15A-15B.

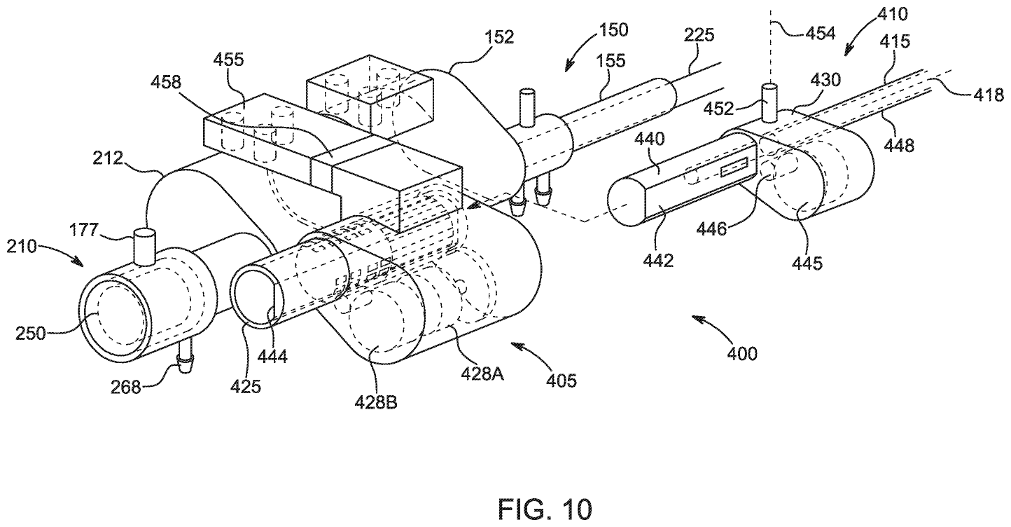

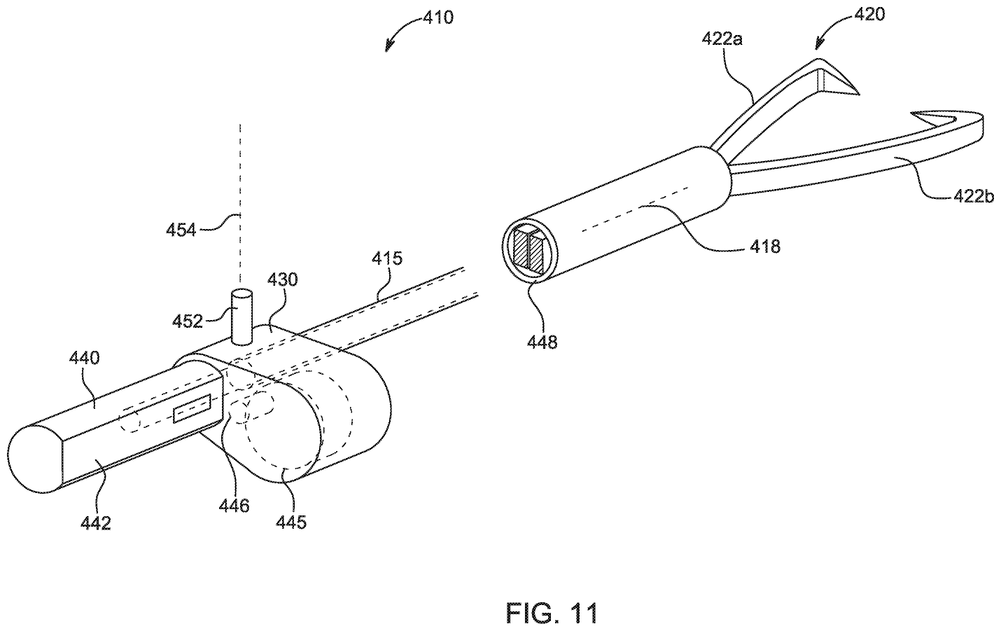

Now turning to FIGS. 10 and 11, another robotic component configured for use with the system 100 of FIG. 1, is shown that comprises a tissue-stabilizing assembly 400 adapted for engaging and gripping the patient's cervix as is known in the art. In any typical intra-uterine procedure using a transcervical approach, the physician uses a tenaculum or forceps to grip and stabilize the patient's cervix to facilitate introduction of an endoscope shaft through the patient's cervical canal CC (FIGS. 16A-16B). In this robotic variation, the approach is similar, with the stabilizing assembly 400 being a robotic assembly. FIG. 10 shows the proximal portions of the endoscopic viewing assembly 140 and resecting device 145, together with the tissue stabilizing assembly 400. The tissue stabilizing assembly 400 again has a drive component 405 that is motorized to drive a tool or tenaculum component 410, shown more fully in FIG. 11. The tenaculum component 410 has an elongate shaft 415 extending about axis 418 to a working end 420 with openable-closeable first and second jaws 422a and 422b (FIG. 11). While FIGS. 10-11 illustrate a working end 420 in the form of a conventional tenaculum. It should be appreciated suction-contact working ends, and other forms of graspers may be used to engage and stabilize a patient's cervix. In the variation shown, the stabilizer drive component 405 is similar to the previously described tool drive component 212 of FIG. 7 in that it again provides two motor drives for both rotational and axial movement of a receiver 425 therein that receives the tenaculum component.

As can be seen in FIG. 10, the drive component 405 carries first and second motors 428A and 428B that are configured to move the receiver 425 both rotationally and axially. The drive systems can be the same as those described in the tool drive component 212. The proximal handle or housing 430 of the tenaculum 410 again includes an extending portion 440 with a keyed surface 442 that cooperates with the keyed surface 444 in the receiver 425. The tenaculum component 410 further carries a motor drive 445 with a suitable gear mechanism such as a worm gear 446 for advancing and retracting the outer sleeve 448 of the shaft assembly 415 as indicated in FIG. 11 to close and open the jaws 422a and 422b. The proximal housing 430 also carries a connector 452 for connecting an electrical cable 454 to the housing 430 to deliver current to the motor 445. In FIG. 10, it can be seen that stabilizer drive component 405 has a bracket 455 for coupling the tissue-stabilizing assembly 400 to the robotic arm 110, for example, with a multiple pin arrangement of the type described previously. In one variation shown in FIG. 10, a middle portion of the bracket 455 of the stabilizer drive component 405 can include a flexible or resilient material 458 to allow for flexing between that stabilizer drive component 405 and the other tools.

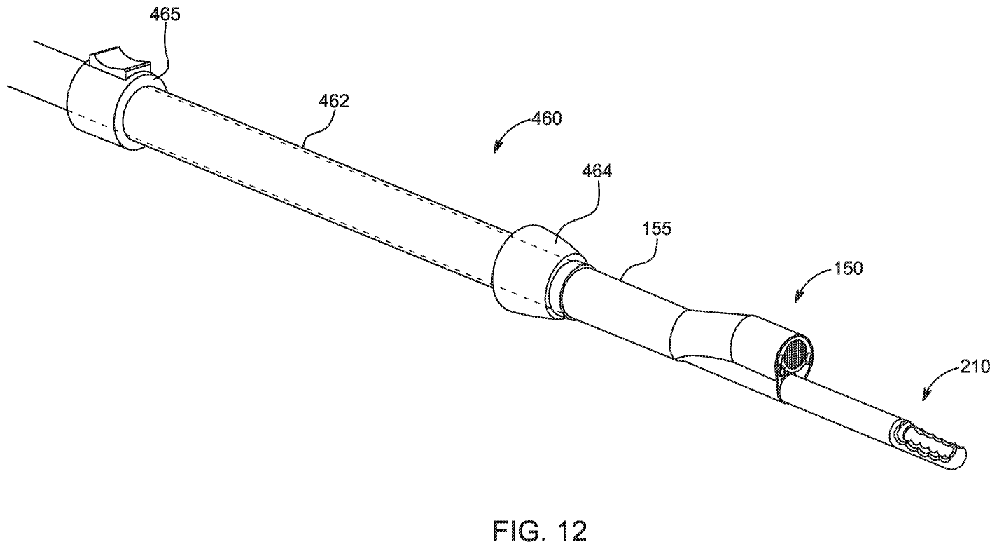

FIG. 12 shows another component of the invention, which comprises a cervical seal 460. Such a cervical seal 460 comprises a concentric sleeve 462 that slides over the shaft 155 of the endoscope 150. The cervical seal 460 carries a tapered distal seal member 464 that can be pushed against the patient's cervix CC and optionally into the cervical canal CC to prevent distention fluid from escaping the uterine cavity through the cervical canal (see FIG. 16B). In this variation, the cervical seal 460 is mounted on the endoscope shaft 155 during manufacturing and is manually moved axially with a locking mechanism 465 at a proximal end of the sleeve 462.

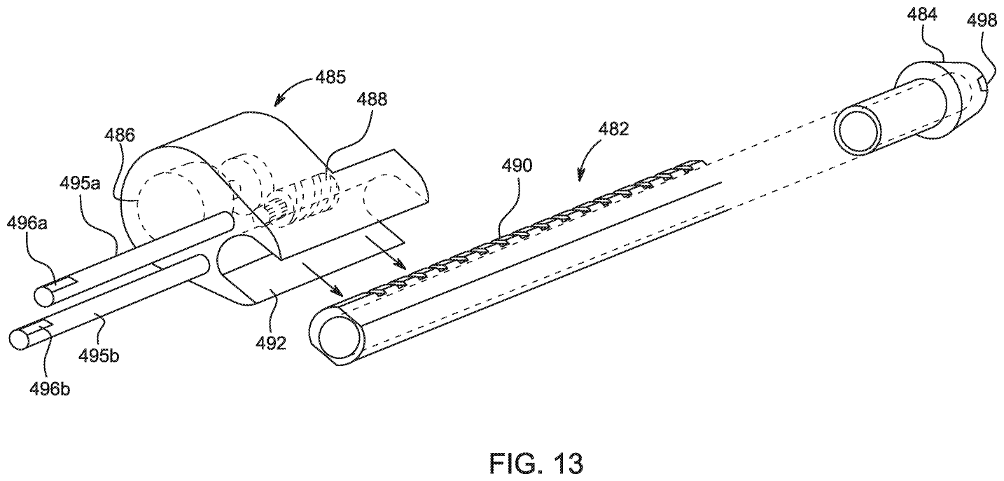

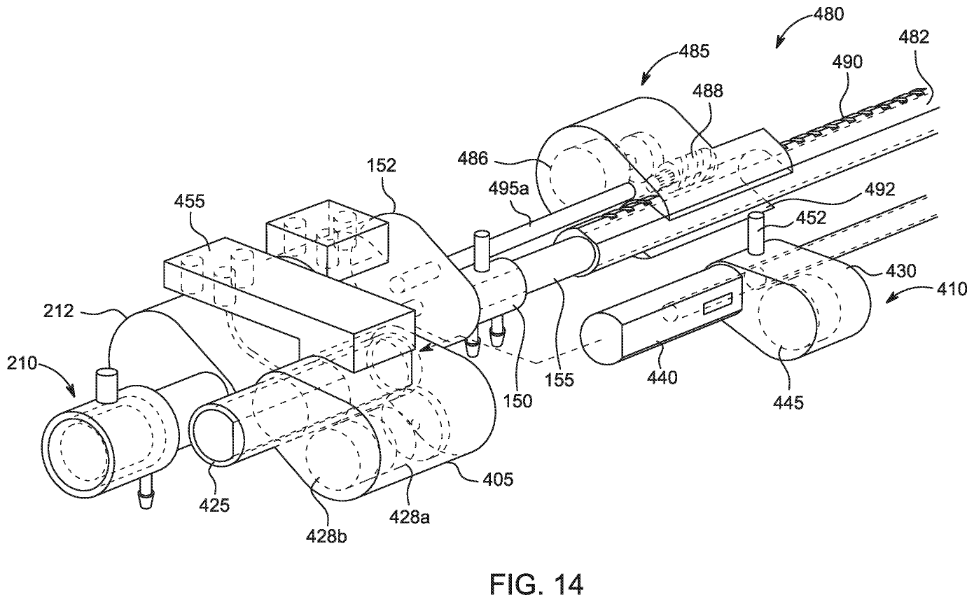

Now turning to FIGS. 13 and 14, a robotically controlled cervical seal 480 is shown that again comprises a sleeve 482 that extends over the endoscope shaft 155 with a distal tapered seal member 484 that contacts the patient's cervix. In this variation, referring to FIG. 14, a seal drive component 485 is provided that is adapted to robotically and automatically move the seal sleeve 482 back and forth to maintain a proper seal. Similar to the other drive components described above, the seal drive component 485 unit has a DC motor 486 that drives a worm gear 488 that engages projecting features 490 on the sleeve 482 to move the sleeve back and forth. In this variation, the drive component 485 has an open-sided slot 492 for receiving the sleeve 482 so that the drive can be mounted over the already assembled endoscope 150 and its drive component 212. In one method of use, the cervical seal sleeve 482 would be advanced manually to engage the cervix, and then the drive component 485 would be positioned in place to engage the seal sleeve 482 for continuous adjustment during a resection procedure using a sensor system described below. The cervical seal sleeve 482 may need an extended range of motion, and the robotic adjustment is best suited for precise shorter axial movements once the seal member 484 is proximate to the patient's cervix. In FIGS. 13-14, it can be seen that the seal drive component 485 has elongated mounting pins 495a and 495b that are adapted to couple the housing 195 of the endoscope drive component 212, as shown in FIG. 14. In this variation, the mounting pins 495a and 495b carry electrical contacts 496a and 496b for engaging cooperating electrical contacts in the endoscope drive housing 195 to carry current to the DC motor 486 in the seal drive component 485.

In one variation, the cervical seal 480 can have one or more sensors 498, as shown in FIG. 13, for sensing contact with the patient cervix, which sends signals to a controller 500 to operate the drive component 485 to move and maintain the seal member 484 in suitable engagement with patient's cervix to prevent leakage of distension fluid. Such a sensor 498 can be any suitable sensor with a wired connection through the electrical contacts 496a and 496b or a wireless connection to the controller 540 or controllers, such as a capacitance sensor, impedance sensor, pressure sensor, light sensor, or the like.

FIG. 14 illustrates the four robotically driven components (endoscopic viewing assembly 140, resecting device 145, tissue-stabilizing assembly 400, cervical seal 480), and the steps of assembling the three components can be understood. In use, as will be described below, the illustrations of the receivers of the drive components may not be drawn to scale, and it can be appreciated that the extendable dimensions of the tools relative to one another may be substantial. FIG. 15 illustrates the working ends of the devices, and it can be understood that the tenaculum shaft 415 is initially configured to be extendable beyond the endoscope working end 160 by up 10 cm to grip the patient's cervix (FIG. 16A). Thereafter, the endoscope shaft 155 is introduced through the patient's cervical canal CC while the clamped tenaculum working end 420 remains stationary so that drive component 152 is configured to extend the endoscope working end 160 a distance D1 in FIG. 15 that is up to 15 cm beyond the tenaculum working end 420 (FIG. 16B). Further, the tool drive component 212 and tubular cutter 210 are configured to extend the working end 240 of the tubular cutter a distance D2 in FIG. 15 that is up to 10 cm beyond the end of the working channel WC and image sensor 162 (FIG. 16C). Also, the cervical seal member 484 requires a large range of axial adjustments over the endoscope shaft 155, where both manual and robotic adjustments are used as described above.

Referring back to FIG. 1, the fluid management system 180 is operatively connected to the endoscope 150 and tubular cutter 210, as is known in the art. The fluid management system 180 includes an inflow peristaltic pump 500A and an outflow peristaltic pump 500B. Inflow tubing 178a has a proximal end coupled to a fluid source 510, such as a saline bag with the distal end of the inflow tubing 178a connected to the Luer fitting 176a (FIG. 4) in an inferior surface of the endoscope handle 154. The inflow pump 500A is adapted to provide a fluid flow through inflow tubing 176a and a flow channel 182 in the endoscope shaft 155 to an outlet 184 in the working end 160 of the endoscope (see FIG. 4). In a variation, the outflow tubing 178b is coupled to Luer fitting 176b on the endoscope 150 (FIG. 4), and a branch 515 of the outflow tubing 178b is coupled to the Luer fitting 268 on the tubular cutter 210 (FIG. 5). A valve or flow diverter 516 may be provided in outflow tubing 178b to select an outflow fluid path from the endoscope 150 or the tubular cutter 210 (FIGS. 1 and 3). The fluid management system 180 thus allows use of the endoscope 150 alone where inflows and outflows are through the endoscope shaft 155 when no cutting device occupies the working channel WC of the endoscope 150. When the tubular cutter 210 is positioned in the working channel WC as shown in FIG. 3, the outflows would flow through the tubular cutter 210 and fitting 268 on its handle 224. The outflow pump 500B thus is adapted to cause fluid flows from a working space to a collection reservoir 525. A tissue trap (not shown) may be provided in the outflow tubing 178b. Independent motor drives are provided to operate the first and second peristaltic pumps 500A and 500B and are controlled by controller 540 or controllers (FIG. 1), which typically are configured to maintain a set pressure in a working space such as a uterine cavity.

Now turning to FIGS. 16A to 16D, a method of the invention in using the robotic system 100 and the various tools, is shown in schematic views in a robotic procedure to resect a fibroid 544 in a patient's uterus 545. The fibroid 544 is shown in a wall of the uterine cavity 550, but it should be appreciated that in other variations of methods, the targeted tissue can be polyps, adhesions, endometrium, and other abnormal tissues as well as fibroids or myomas. In FIG. 16A, it can be understood that the user has actuated various segments of the robotic arm 110 to align the endoscope shaft 155 and tenaculum shaft 415 with the external os 552 of the patient's cervix 554 after being advanced into the patient's vagina 556, which may be held open with a suitable speculum device (not shown). FIG. 16A shows that under endoscopic vision, the endoscope 155 and tenaculum jaws 422a and 422b are robotically manipulated axially and rotationally as needed and then closed to grasp the cervix 554 to thereby stabilize the cervix.

FIG. 16B illustrates a subsequent step of the method wherein the user operates the robotic arm 110 to advance the endoscope working end 160 in its reduced cross-section or insertion profile through the cervical canal CC into the uterine cavity 550. The fluid management system 180 is typically actuated to provide fluid inflows through the endoscope 150 to facilitate introduction of the working end 160 through the cervical canal CC. The S-shaped end of the endoscope working end 160 may be rotated to position it optimally relative to the tenaculum jaws for introduction into the cervical canal CC. FIG. 16B further illustrates the step of manually advancing the seal sleeve 482 and seal member 484 toward the cervix 554 and cervical canal CC.

FIG. 16C illustrates the seal sleeve 482 and seal member 484 fully advanced to contact the cervix 554, either manually or with drive member 485, to prevent distension fluid from leaking outwardly from the cervical canal CC. FIG. 16B further shows the user actuating the tool drive component 212 to advance the shaft 225 of the tubular cutter 210 through the working channel WC in the endoscope shaft 155 into the uterine cavity 550, which expands the distal portion of the working channel WC as described above. FIG. 16C further shows the robotic arm 110 be actuated to move the distal ends 160 of the endoscope 150 and the distal end 240 of the tubular cutter 210 in the direction of plane X to interface with the surface of the fibroid 544.

FIG. 16D illustrates the user operating the robotic arm 110 further to tilt the working end 240 and cutting windows of the tubular cutter 210 into the fibroid 544 to resect and remove tissue. It should be understood that this step of the method of resecting tissue can include moving the working end 240 of the cutter 210 axially and/or rotating the working end slightly back and forth to resect the fibroid 544. Further, the user may rotate the shaft 155 of the endoscope to reposition the image sensor 162 and LED's, as shown in FIG. 6, to acquire the best view of the surface of the fibroid 544 that is being resected. During the resection procedure, the seal drive component 485 can operate to adjust seal 484 to maintain contact with the cervix to prevent fluid leakage, as described above.

While FIG. 16D illustrates the step of a method of resecting a fibroid. It should be further appreciated that operating parameters of the tubular cutter 210 and the robotic system 100 can be controlled or automated during the resection by the controller 540 or multiple controllers of the robotic system 100. In a variation, for example, the fluid management system 180 can be controlled by the user from the user input interface 115 during resection, which includes adjusting any operating parameter of the fluid management system. Operating parameters of the fluid management system include (i) adjusting the set pressure in the uterine cavity, (ii) increasing fluid inflow temporarily as a flush mechanism to clear resected debris or blood from the viewing field, (iii) increasing fluid pressure to a selected level for a selected time interval as a tamponade, and (iv) adjusting the fluid deficit level and any warnings or system shut-down associated with a fluid deficit. The user can also adjust operating parameters of the endoscope 150 from the user input interface 115, which includes (i) adjusting light from the LEDs and (ii) capturing images or videos of the procedure.

The user can also adjust operating parameters of the tubular cutter 210 at the user input interface 115, which includes (i) adjusting rotational speed of the inner cutting sleeve 144, and (ii), in some variations, adjusting oscillation and/or translation of the inner cutting sleeve 144 within the outer sleeve 146 during use. As is known in the art, tubular cutters may use rotating cutting sleeves, reciprocating cutting sleeves, or rotating and axially translating cutting sleeves.

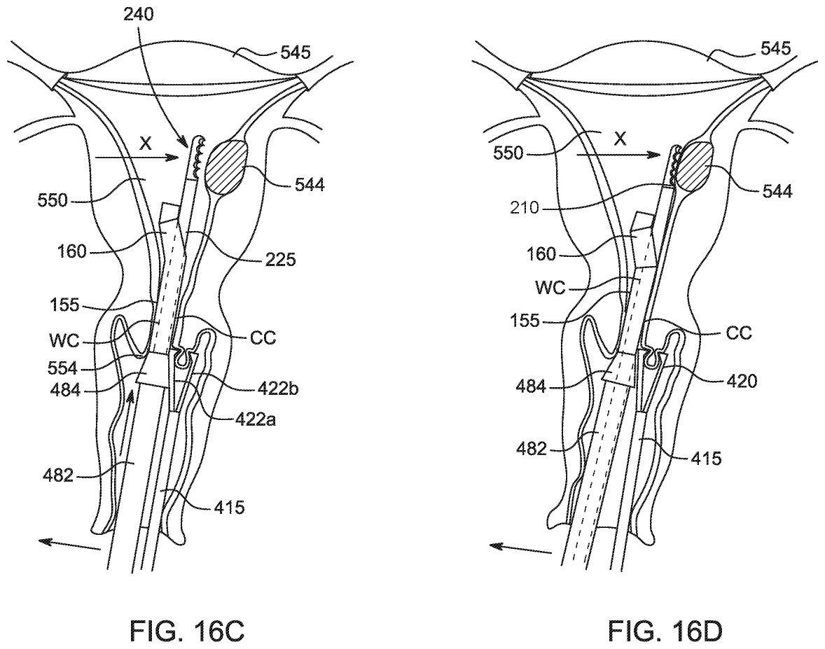

In another variation of a method of resecting tissue, referring to FIG. 6 and FIG. 17, the controller 540 or controllers of the robotic system 100 can automate parameters of a resecting interval in a preset “resecting plan” in which actuation of drive components of the resecting device and the motors of the robotic arm 110 move the working end 160 of the tubular cutter 210 in preset movements while operating to efficiently resect tissue. For example, in FIG. 6, the user can select via user input interface 115 a resecting plan “RP1” in which the inner cutting sleeve 144 is operated at a selected RPM between 1,000 RPM and RPM while contemporaneously, the drive component 212 moves the working end 160 axially back and forth (direction AX1 in FIG. 6) at a selected rate from 0.1 cm/sec to 1.0 cm/sec and for a selected reciprocating distance of 0.1 cm to 2 cm. The resecting plan RP1 can operate for a preset number of seconds, for example, from 5 seconds to 30 seconds, or can be initiated and terminated by user input. Another preset resecting plan (not shown) can operate the inner cutting sleeve 244 at an RPM range described above while contemporaneously actuating the drive component 212 to rotate working and 160 of the tubular cutter 210 from to 180o degrees at a selected rate ranging between 10o/sec and 90o/sec. Another variation can consist of a preset resecting plan, “RP2,” as shown in FIG. 17, which combines both reciprocation and rotation within the parameters described above to resect tissue for any pre-selected time interval or by initiation and termination by the user. FIG. 17 depicts discrete, sequential axial and rotational movements, but a zig-zag movement is possible with contemporaneous axial and rotational movements. At the same time that the working end 160 of the tubular cutter 210 is being used in any preset resecting plan, the user can rotate the working end 160 of endoscope 150 (FIGS. 6 and 17) to optimize the viewing of the resection of tissue. In any of the resecting plans above, the user would further operate the robotic arm to move the cutter working end 240 in any direction X, Y, or Z (FIG. 3) to optimally engage the targeted tissue.

In FIG. 1, it can be seen that the console 112 with user input interface 115 is connected by a cable 580 to a controller 540 and to a connector 582 at the base of the robotic arm 110. The various tools and drive units 140 and 145 also can connect by cables, for example, cables 186 and 278 to the connector 582 to provide for electrical power transmission and electrical signaling between all the tools, the console 112 and the controller 540 or controllers. While electrical cables are shown, it should be appreciated that electrical signaling also may be wireless.

In another variation, referring back to FIGS. 4 and 5, the extending portion 174 of the housing 154 of endoscope 150 that is received by the receiver 200 can carry one or more load sensors 590 or force sensors around the circumference of the housing that interface with the receiver 200 to sense forces being applied to the working ends 160, 240 and shafts 155, 225 of the endoscope 150 and tubular cutter 210 during a resecting procedure (FIG. 16D). Signals from the force sensor(s) 590 are received by the controller 540, and a control algorithm can continuously monitor the forces and the direction of forces on the shafts 155 and 225 of the devices. In a variation, excess forces, such as bending forces, on the shafts 155 and 225 can provide an audible signal or a visual signal that forces are exceeding a preset level, or the control algorithm can stop operation of the resecting device or automatically initiate a movement of the robotic arm in a suitable direction to reduce forces on the shafts 155 and 225 of the endoscope 150 and the tubular cutter 210. In another variation, any preset resecting plan above (e.g., RP1 of FIG. 6; RP2 of FIG. 17) can include a resection plan that actuates the robotic arm 110 to move the working end 240 of the cutter 210 in a pre-selected range of X, Y, and Z movements (see FIG. 3) wherein the force-sensing algorithm further automatically controls and maintains a pre-selected pressure on the working end 240 as it engages and resects tissue. It should be appreciated that such force sensors 590 can be positioned in any tool housing or any cooperating drive unit where the sensing forces on the tenaculum 410 also may be useful.

In general, a robotic system 100 of the invention comprises a surgical robotic arm 110 with a base and a plurality of arm segments that rotate or translate relative to a plurality of axes, wherein at least one distal segment of the robotic arm is adapted for detachably coupling to a single-use endoscopic viewing system 140, a resecting device 145 and a tissue-stabilizing system 400 that all can be operated with robotic drive mechanisms to move the tools from a remote user input interface 115.

A method of the invention for treating targeted tissue in a patient's uterine cavity comprises providing a robotic arm having a plurality of arm segments to provide movement of the robotic arm relative to a plurality of axes, wherein a distal arm segment carries a single-use endoscope with an elongate shaft having a working channel therein, wherein the endoscope shaft has a first insertion profile having a non-expanded shape and a second expanded shape by introduction of a shaft of a treatment tool through the working channel, and thereafter (i) operating the robotic arm to introduce the endo scope shaft in its first insertion profile through the patients cervical canal into a uterine cavity, (ii) operating the robotic arm to introduce the shaft of the treatment tool through the working channel thereby expanding the working channel to its second expanded shape and moving the working end to its expanded working profile, and (iii) treating the targeted tissue in the uterine cavity with the working end of the treatment tool. Additionally, robotic stabilizing carried by the robotic arm ac be used to stabilize the patient's cervix before introducing the endoscope through the patient's cervical canal.

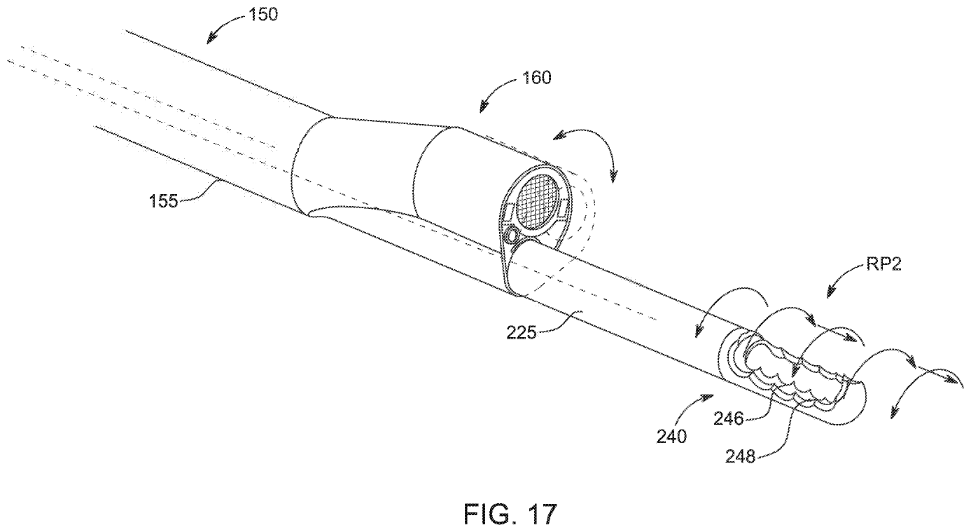

Now turning to FIGS. 18-20, another variation of a treatment tool is shown, which comprises a retractor 600 with an elongate shaft 605 that is configured for introduction through the working channel WC of the endoscope 150 as described above. In this variation, the tubular cutter 610 is similar to the cutters described above, except the cutter shaft 612 and working end 614 have a smaller diameter to cooperate with the retractor shaft 605 so that the combination of the retractor shaft 605 and cutter shaft 612 can be slidably and rotationally disposed in the working channel WC of the endoscope 150. Thus, the working end 614 of the tubular cutter and the working end 615 of the retractor 600 can be positioned adjacent or aside each other in the working channel WC of the endoscope.

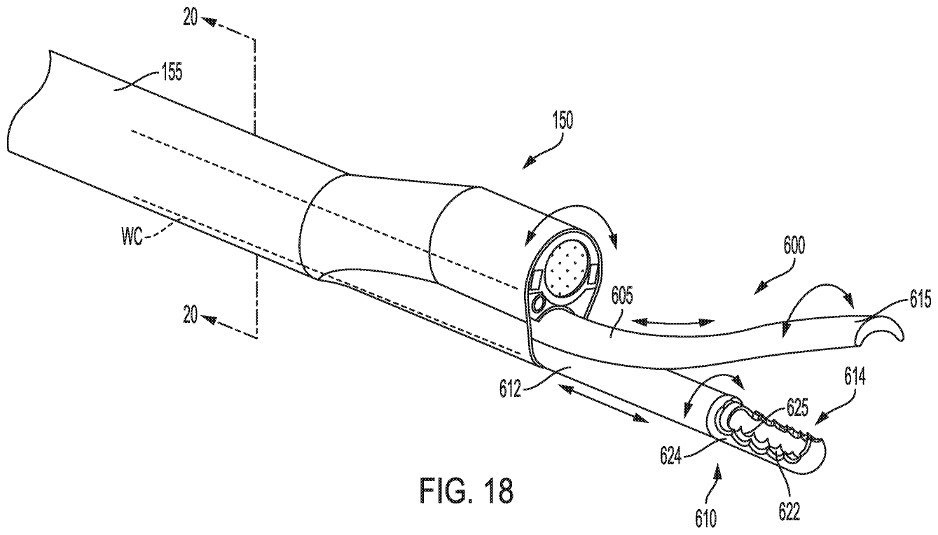

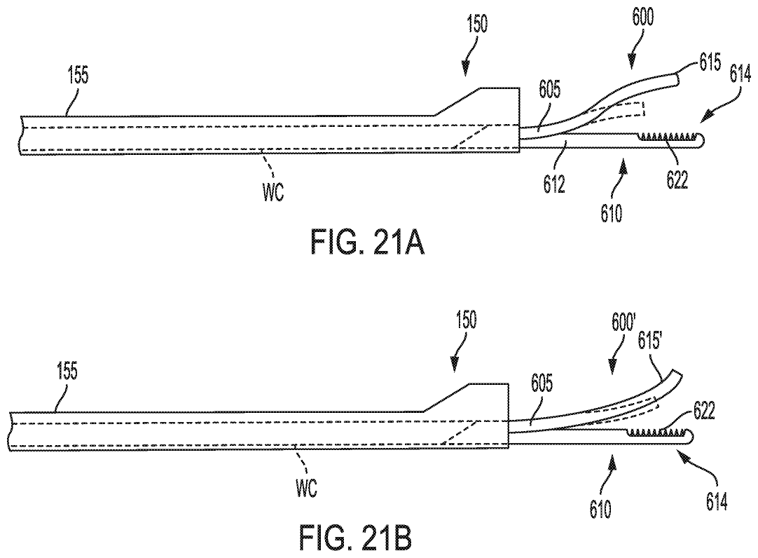

Various types of retractors are potentially useful with FIGS. 18-20 showing an aspiration-based retractor 600 that can engage tissue by means of its working end 615 configured with one or more suction ports 616, as can be seen in FIG. 19 disposed in a surface 620 of the working end that can face the cutting windows 622 (cutting windows in outer sleeve 624 and rotating inner sleeve 625) of the tubular cutter. The purpose of a retractor 600 or retraction system of the invention is to contact, engage, grasp, or stabilize targeted tissue that can then be resected with the tubular cutter 610. In many cases, the targeted tissue may be a pedunculated polyp 626, as shown in FIG. 22 that consists of a volume of tissue that is not stable and is difficult to engage with the cutting windows 622 of the tubular cutter 610. As can be understood, the resection of a polyp 626 (FIG. 22) differs from the resection of a fibroid 544 in a uterine wall, as shown in FIGS. 16A-16D.

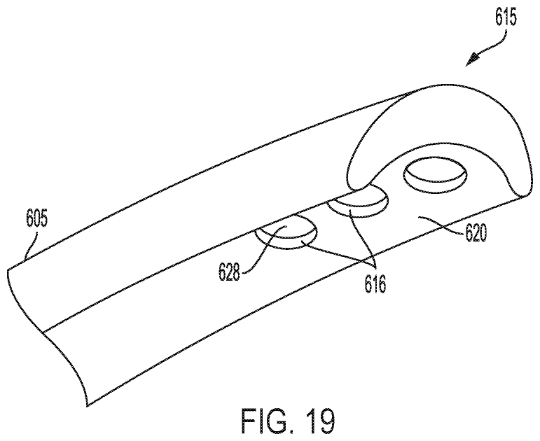

As can be seen in FIGS. 19 and 20, the suction ports 616 communicate with an interior channel 628 in the retractor shaft 605 that, in turn, communicates with the negative pressure source comprising the outflow pump 500B as described in previous embodiments. The one or more suction ports 616 are adapted to suction the polyp 626 against the surface 620 of the retractor 600, and any distention fluid aspirated into the retractor will be carried by the outflow pump 500A to the fluid collection reservoir 525 (FIG. 1).

FIG. 20 illustrates a cross-section of the retractor shaft 605 and the tubular cutter shaft 612 (outer sleeve 624, inner sleeve 625) disposed within the working channel WC, which comprises the interior channel of sleeve 630 in the medial region of the endoscope shaft 155. In FIG. 20, it can be understood that the combination of the retractor shaft 605 and the tubular cutter shaft 612 can be rotated within the working channel WC. Further, the retractor shaft 605 is dimensioned so that it can be rotated to some extent relative to the shaft 612 of the tubular 610 cutter within the working channel WC, for example, up to 30o, as shown by arc C in FIG. which indicates rotation away from the maximum radial spacing between the cutter shaft 612 and interior surface 632 of working channel sleeve 630.

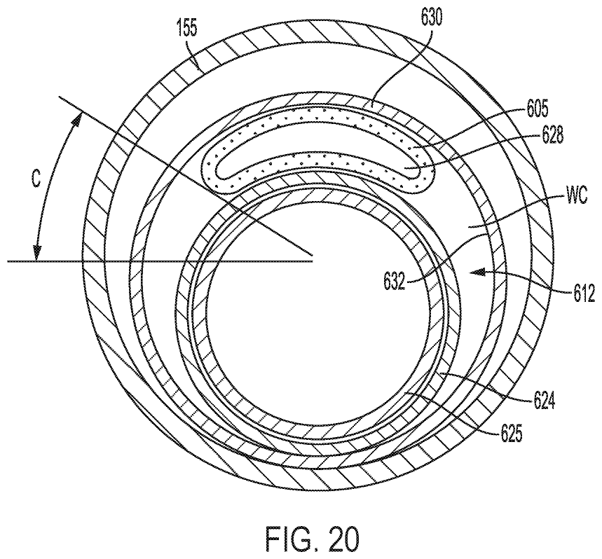

Now referring to FIGS. 18 and 21A, it can be understood that the working end 615 of the retractor shaft 605 comprises a resilient polymer material that has a repose or non-tensioned shape that is curvilinear. In FIGS. 18 and 21A, the resilient region comprises the working end 615 of the retractor shaft 605 and has an S-shaped curve. It can be understood that retraction of the resilient region of the shaft 605 into the working channel WC will straighten the resilient working end 615. Further, it can be understood that distally advancing the working end 615 of the retractor shaft 605 outward from the working channel WC causes it to transition through a range of shapes and a range of distances apart from the rigid shaft 612 and cutting windows 622 of the tubular cutter 610. Also, it can be easily understood that relative axial movement of the retractor shaft 605 and tubular cutter shaft 612 can adjust the dimensions between the retractor working end 615 and the cutting windows 622 of the tubular cutter. In this way, the engaged tissue or polyp 626 can be moved relative to the cutting windows 622. FIG. 21B shows another variation of resilient retractor 600′ with retractor shaft 605′ having a working end 615′ that has a single curvature, for example, a C-shaped curve, which otherwise is similar to the S-shaped working end 615 of the retractor 600 of FIGS. 18, 19 and 21A. It should be appreciated that various curvilinear shapes can be provided in retractor working ends, and during a resection procedure, it will be possible to insert and withdraw various inexpensive retractors with differently shaped working ends, which may be optimized for the anatomy of the targeted tissue.

Now turning to FIG. 22, a method of using the retractor 600 is shown schematically where the endoscope 150 and cervical seal member 484 are in place as described in the previous method illustrated in FIGS. 16C-16D. In this method, it can be seen that the retractor working end 615 is advanced outwardly from the endoscope 150, and suction is applied through the retractor to contact, engage and stabilize the polyp 626. Thereafter, the tubular cutter 610 is advanced into position to resect the polyp. The cutting windows 622 of the tubular cutter 610 can be moved toward the polyp, and further, the retractor working end 615 can be retracted to move the engaged polyp 626 toward the cutting windows 626 of the tubular cutter. Further, the working end 614 of the tubular cutter 610 can be retracted and rotated to engage and resect the polyp 626. At the same time, the working end of the endoscope 150 can be rotated relative to the retractor 600 and tubular cutter 610 for optimal viewing of the resection procedure.

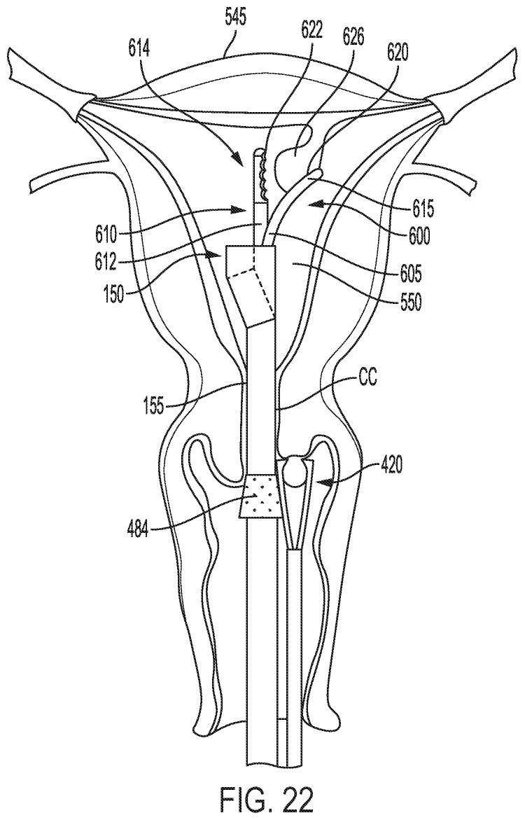

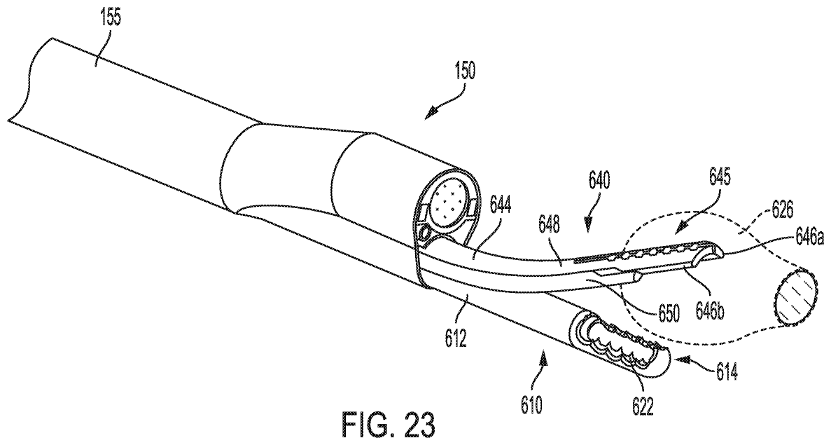

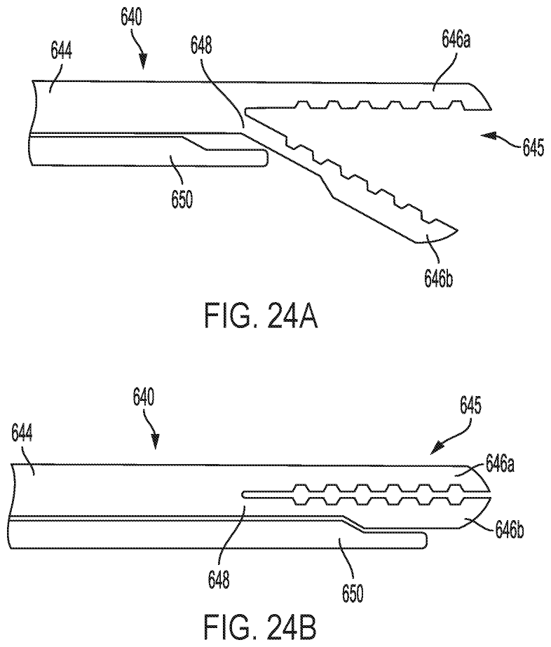

Now turning to FIGS. 23-25, another variation of retractor 640 with shaft 644 is shown that is similar to the previous embodiment in that it has a resilient polymer working end 645 with a curvilinear shape and is adapted to engage tissue such as a polyp. In this variation, the retractor working end 645 comprises a jaw structure with first and second jaws 646a and 646b that is configured for opening and closing to grasp tissue such as a polyp 626. In the variation of FIGS. 23-25, the jaw structure comprises first and second jaws 646, 646b of a resilient material with a living hinge element 648 at the proximal end of the second jaw 646b. In this variation, the first jaw 646a is not moveable, and the second jaw 646b has a repose position in the jaw-open configuration of FIG. 24A. The jaws are moved toward a jaw-closed position of FIG. 24B by the axial extension of an actuator member 650. FIG. 25 is a sectional view of the retractor shaft 644 in the working channel WC adjacent to the outer and inner sleeves 624, 625 of the tubular cutter. It can be seen that the slidable actuator member 650 has a key feature 652 that slides in an axial receiving channel 655 in retractor shaft 644. It can be appreciated that only a light grip on a targeted polyp 626 may be sufficient to stabilize the polyp for resection. The jaws 646, 646b only require the low gripping force, as shown schematically in FIG. 23. In other aspects, the grasping retractor, the FIGS. 23-25 is similar to the previous aspiration-based retractor 600 of FIGS. 18-20. In the variation of FIG. 23, the retractor 640 again may be operated manually or can be coupled to one or more motor drives to move the retractor rotationally in combination with the tubular cutter or axially relative to the tubular cutter and also robotically move the actuator member 650 for closing and opening the jaws 646, 646b.

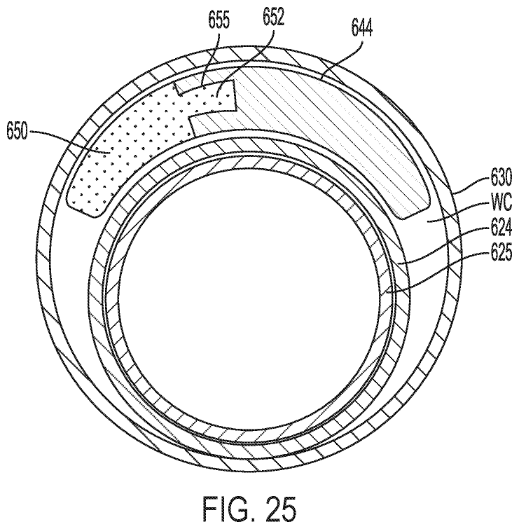

FIG. 26 illustrates another variation of a retractor 660 that is similar to that of FIG. 18, in which an aspiration-based retractor has an interior channel 662. In this variation, the retractor 660 has a resilient curvilinear working end 665 together with an actuator member 668 in the form of a stiff loop-shaped member, as shown in FIG. 26, that can be axially moved within the interior channel 662 to adjust the shape of the working end 665. Thus, the shape of the working in 665 can be adjusted by either axial extension or retraction of the actuator member 668 in the interior channel 662 or the advancement or retraction of the working end 665 from the working channel WC. Again, the axial movement of the actuator member 668 and/or the working end 665 can be accomplished manually or robotically.

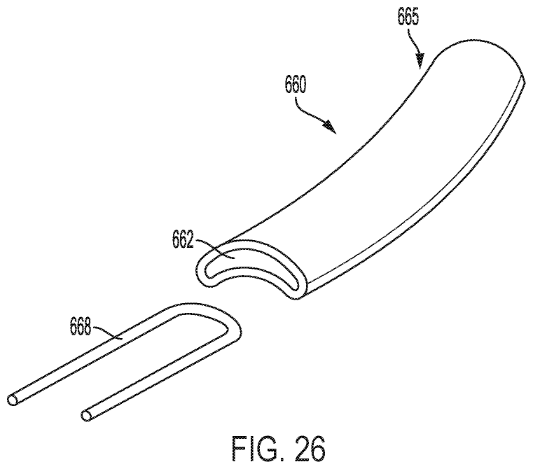

FIG. 27 illustrates a variation of retractor 680 with a shaft 682 and a distal jaw structure 684 having a first jaw 685a and a second jaw 685b with an interior channel 686 that receives an axially moving actuator 688 for moving in second jaw 285b from an open position to a closed position. The resilient material of the jaw structure 684 and living hinge 689 again has a repose or non-tensioned shape with the jaws open, as shown in FIG. 27. It can be appreciated that a similar jaw structure can have dual actuating members in dual interior channels wherein both jaws are then moveable by movement of the actuating members.

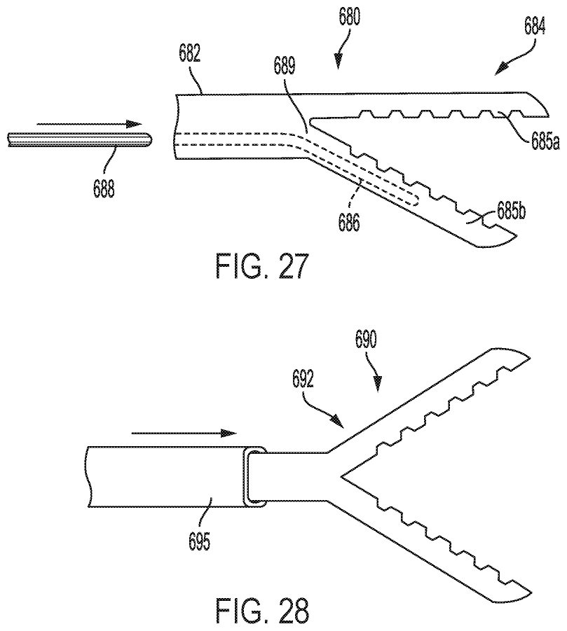

FIG. 28 shows another variation of a retractor 690 with a resilient jaw structure 692 that is configured for closing with axial movement of an exterior sleeve 695.

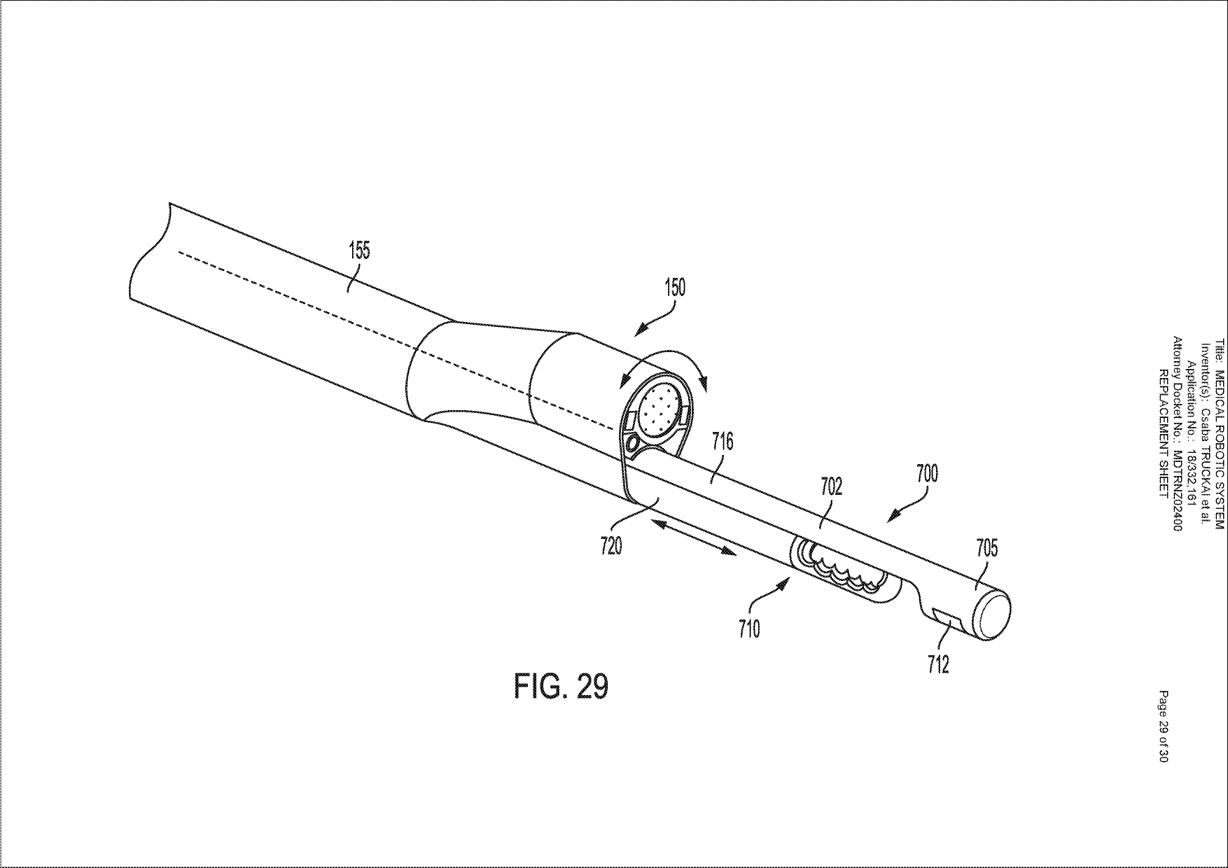

Now turning to FIG. 29, another variation of a treatment tool 700 with an elongated shaft 702 and a working end 705 is shown where the tool again is configured for use in combination with a tubular cutter 710. In this variation, the treatment tool 700 carries an ultrasound transducer 712 in the working end 705, which has an expanded cross-section relative to the proximal and medial shaft portions 716. It should be appreciated that the expanded cross-section working end 705 can comprise other functional devices such as a retractor, grasper, biopsy device, RF device, or the like. As can be seen in FIG. 29, the medial portion 716 of the tool shaft 702 is configured for positioning in the working channel WC together with the shaft 720 of the tubular cutter 710. However, the working end 705 of the treatment tool 700 can have a cross-section that equals the cross-sectional dimension D of the working channel WC. In other words, the working ends of the tools can be stacked and axially spaced apart, where the variations of FIG. 18-21 positioned the working ends of both tools aside from one another when retracted into the working channel WC. The shaft 702 of the tool 700 again may comprise a resilient material having a curved repose shape to position the working end 705 away from the tubular cutter.

Referring to FIG. 29, the ultrasound transducer 712 can be used to image the targeted tissue, such as a fibroid 544, as in FIGS. 16A-16D, to observe the depth of resection in the uterine wall. Such ultrasound images, when displayed on a video display, can allow the operator to observe the layers of different tissues in the uterine wall, such as the serosa, to prevent resecting too close to the serosa. Thus, the use of such an ultrasound transducer 712 can assist the physician in preventing any perforation of the uterine wall.

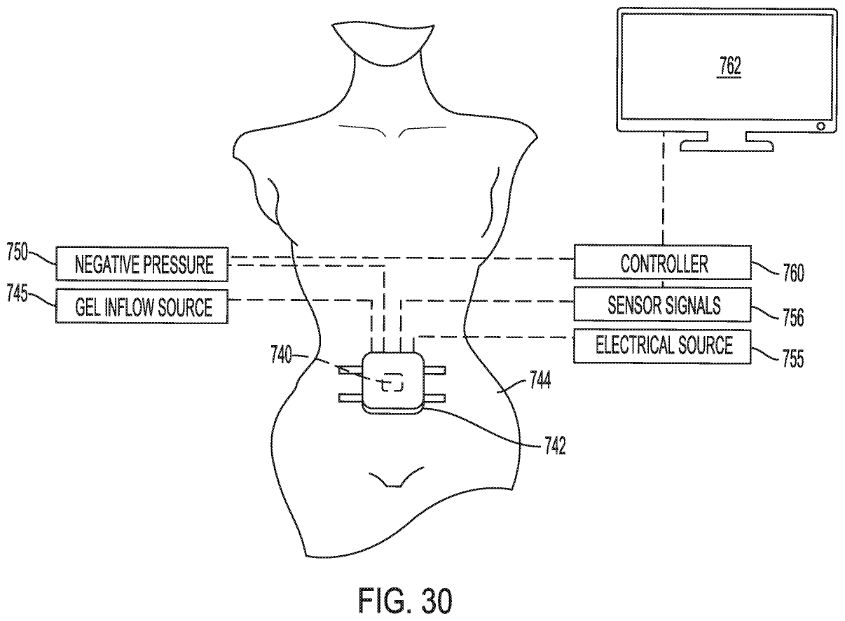

Now turning to FIG. 30, another aspect of the invention comprises another variation of using an ultrasound transducer 740 to monitor a resection procedure. In the variation of FIG. 30, one or more ultrasound transducers 740 are carried in a housing 742 positioned on the exterior of the patient's body 744. In this variation, the exterior ultrasound transducer or transducers can image the uterine wall (see FIGS. 16A-16D), and the physician can observe the different tissue layers in the uterine wall, such as the serosa, to prevent cutting too deep when resecting a fibroid. In the variation of FIG. 30, the housing 742 carrying the ultrasound transducer or transducers 740 is positioned on the patient's skin above the uterine cavity. The housing 742 can be affixed to the patient's body 744 by any suitable means, such as adhesives, suction devices, bandages, belts, etc. In one variation, a pressurized source of ultrasound gel 745 communicates with the housing 742 to deliver additional gel to the interface between the transducer 740 and the patient's skin during the course of a medical procedure. FIG. 30 further shows a negative pressure source 750 that may be used to suction the housing 742 against the patient's skin. FIG. 30 further illustrates that the ultrasound transducer 740 is powered by an electrical source 755, and sensor signals 756 are sent by cable to a controller 760 and video display 762. In another variation, it should be appreciated that the ultrasound images from the exterior transducer 740, as in FIG. 30, and the images from the intra-cavity ultrasound transducer 712 of FIG. 29 can both be displayed on the video display 762. By having images from both the intra-cavity and exterior transducers 712, 740, the physician may be allowed better views of the layers of the uterine walls to prevent perforation.

Additional features of the ultrasound system and controller 760 may be used to control, modulate or terminate actuation of the resection devices or provide an alarm signal when control algorithms observe that the cutter is within a pre-selected distance from the exterior wall of the uterus.

The various retractors and treatment tools illustrated above or designed to minimize cross-sectional shapes and limit the number of moving parts. However, it should be appreciated that any of the treatment tools can be articulated with pull wires, slotted sleeves, nitinol sleeves, and other mechanisms known in the art. Further, the tubular cutter can have an articulating working and with a flexible drive in the inner cutting sleeve where pull wires, or slotted sleeves can be used to articulate the working end of the cutter.

Although particular embodiments of the present invention have been described above in detail, it will be understood that this description is merely for purposes of illustration, and the above description of the invention is not exhaustive. Specific features of the invention are shown in some drawings and not in others, and this is for convenience only, and any feature may be combined with another in accordance with the invention. A number of variations and alternatives will be apparent to one having ordinary skills in the art. Such alternatives and variations are intended to be included within the scope of the claims.

Particular features that are presented in dependent claims can be combined and fall within the scope of the invention. The invention also encompasses embodiments as if dependent claims were alternatively written in a multiple dependent claim format with reference to other independent claims.

It is important to note that where possible, aspects of the various described embodiments or the embodiments themselves can be combined. Where such combinations are intended to be within the scope of this disclosure. All references, including publications, patent applications, and patents, cited herein are hereby incorporated by reference to the same extent as if each reference were individually and specifically indicated to be incorporated by reference and were set forth in its entirety herein.

What is claimed is:

1. A medical robot system for use in intrauterine procedures, comprising:
a robotic arm having a plurality of moveable arm segments;
an endoscope detachably coupled to a distal segment of the robotic arm, the endoscope having an elongate endoscope shaft with a working channel therein, the elongate endoscope shaft extending about a longitudinal axis to a distal end carrying an image sensor;
a resecting device detachably coupled to the distal segment, the resecting device having an elongate resecting shaft configured for introduction through the working channel of the elongate endoscope shaft; and
a retractor having an elongate retractor shaft configured for introduction through the working channel of the elongate endoscope shaft.

2. The medical robot system according to claim 1 further comprising a controller configured to operate at least one of the endoscope, resecting device and retractor.

3. The medical robot system according to claim 2 further comprising a motor drive configured to move the elongate endoscope shaft at least one of axially and rotationally relative to its longitudinal axis.

4. The medical robot system according to claim 2 further comprising a motor drive configured to move the elongate resecting shaft at least one of axially and rotationally relative to its longitudinal axis.

5. The medical robot system according to claim 2 wherein the controller is adapted to actuate a motor driven cutting member of the resecting device.

6. The medical robot system according to claim 2 further comprising a motor drive configured to move the elongate retractor shaft at least one of axially and rotationally relative to its longitudinal axis.

7. The medical robot system according to claim 2 wherein the controller is adapted to actuate a negative pressure source in communication with the retractor.

8. The medical robot system according to claim 2 wherein the controller is adapted to actuate a motor drive to move a jaw structure of the retractor between open and closed positions.

9. The medical robot system according to claim 2 wherein the controller is adapted to actuate a negative pressure source that communicates with an interior channel and suction ports in the retractor.

10. A method of treating tissue in a patient's uterine cavity, comprising:
providing a robotic arm with an elongated electronic endoscope detachably coupled to a distal segment of the robotic arm, and a resecting device and retractor configured for introduction through a working channel of the elongated electronic endoscope;
introducing the elongated electronic endoscope trans-cervically into the patient's uterine cavity;
advancing the resecting device and retractor through the working channel of the elongated electronic endoscope into the patient's uterine cavity;
actuating the retractor to engage and stabilize targeted tissue; and
actuating the resecting device to resect the targeted tissue.

11. A surgical retractor combination adapted for engaging targeted tissue, comprising:
an endoscope having an elongate endoscope shaft with a working channel therein, the elongate endoscope shaft extending about a longitudinal axis to a distal end carrying an image sensor; and
a retractor having an elongate retractor shaft configured for introduction through the working channel in the elongate endoscope shaft;
wherein a working end of the retractor comprises a resilient material having a curvilinear repose shape when extended outwardly from the working channel and is capable of tensioned axial straight shape when retracted into the working channel.

12. The surgical retractor combination according to claim 11 wherein the working end of the retractor has repose shape in a form of an “S” curve.

13. The surgical retractor combination according to claim 11 wherein the working end of the retractor has repose shape in a form of an “C” curve.

14. The surgical retractor combination according to claim 11 wherein the elongate retractor shaft has an interior channel therein.

15. The surgical retractor combination according to claim 14 further comprising a negative pressure source communicating with the interior channel and suction ports in the working end of the retractor.

16. The surgical retractor combination according to claim 15 further comprising an axially moveable deflecting member configured for axially movement in the interior channel to adjust a shape of the working end of the retractor.

17. The surgical retractor combination according to claim 11 wherein the working end of the elongate retractor shaft has a jaw structure configured to open and close.

18. The surgical retractor combination according to claim 17 wherein the jaw structure is adapted for closing by an actuator member that is configured to be axially moveable.

19. The surgical retractor combination according to claim 18 wherein the actuator member is adapted to slidably contact an exterior surface of the jaw structure.

20. The surgical retractor combination according to claim 18 wherein the actuator member is adapted to slidably contact a surface of an interior channel of the jaw structure.

21. The surgical retractor combination according to claim 11 wherein the elongate retractor shaft has an axially concave surface for nesting with a resection device shaft of a resection device.

22. A method of treating targeted tissue in a patient's uterine cavity, comprising:
providing a robotic arm with an elongated electronic endoscope detachably coupled to a distal segment of the robotic arm, and a resecting device an ultrasound device configured for introduction through a working channel of the elongated electronic endoscope;
introducing the elongated electronic endoscope trans-cervically into the patient's uterine cavity;
advancing the resecting device and ultrasound device through the working channel of the elongated electronic endoscope into the patient's uterine cavity;
actuating the ultrasound device to image the targeted tissue; and
actuating the resecting device to resect the targeted tissue.

23. A method of treating targeted tissue in a uterine cavity of a patient's body, comprising:
providing a robotic arm with an elongated electronic endoscope detachably coupled to a distal segment of the robotic arm, and a resecting device configured for introduction through a working channel of the elongated electronic endoscope;
positioning at least one ultrasound transducer on an exterior of the patient's body to image the targeted tissue;
introducing the electronic endoscope trans-cervically into the uterine cavity;
advancing a resecting device and an ultrasound device through the working channel of the elongated electronic endoscope into the uterine cavity; and
actuating the resecting device to resect the targeted tissue while contemporaneously observing ultrasound images of the targeted tissue.
